(19) United States
(12) Patent Application Publication
(10) Pub. No.: US 2024/0157011 A1
(43) Pub. Date: May 16, 2024

(54) DEVICE FOR TREATMENT WITH ACTIVATED OXYGEN AND METHOD FOR TREATMENT WITH ACTIVATED OXYGEN

(71) Applicant: CANON KABUSHIKI KAISHA, Tokyo (JP)

(72) Inventors: TAKUMI FURUKAWA, Shizuoka (JP); MASAKI OZAWA, Shizuoka (JP); KAZUHIRO YAMAUCHI, Shizuoka (JP); KENJI TAKASHIMA, Kanagawa (JP); MOTOTERU GOTO, Shizuoka (JP); SHOTA KANEKO, Tokyo (JP)

(21) Appl. No.: 18/419,684

(22) Filed: Jan. 23, 2024

Related U.S. Application Data
(63) Continuation of application No. PCT/JP2022/028384, filed on Jul. 21, 2022.

(30) Foreign Application Priority Data
Jul. 30, 2021 (JP) 2021-126211
Jul. 14, 2022 (JP) 2022-112933

(51) Int. Cl. A61L 2/26; A61L 2/10; A61L 2/20

(52) U.S. Cl. CPC A61L 2/26; A61L 2/10; A61L 2/202; A61L 2202/11; A61L 2202/122

(57) ABSTRACT

A treatment device using active oxygen, the treatment device comprising: an ultraviolet transmission member; and a first chamber and a second chamber adjacent to each other separated by the ultraviolet transmission member, wherein the first chamber is capable of accommodating a treatment target and comprises an ozone generator, the second chamber comprises an ultraviolet light source, the ultraviolet light source is capable of irradiating a surface of the treatment target, which is accommodated in the first chamber, with ultraviolet light, and a distance between the ultraviolet transmission member and the surface of the treatment target can be adjusted to 10 mm or less.

### CROSS-REFERENCE TO RELATED APPLICATION

This is a continuation of International Application No. PCT/JP2022/028384, filed on Jul. 21, 2022, and designated the U.S., and claims priority from Japanese Patent Application No. 2021-126211 filed on Jul. 30, 2021, and Japanese Patent Application No. 2022-112933 filed on Jul. 14, 2022, the entire contents of which is incorporated herein by reference.

### BACKGROUND OF THE INVENTION

### Field of the Invention

The present disclosure is directed to a treatment device using active oxygen and a treatment method using active oxygen.

### BACKGROUND ART

Ultraviolet light and ozone are known as means for sterilizing articles and the like. Since there is a problem in that sterilization by ultraviolet light is limited to only a part of an object to be sterilized which is irradiated with ultraviolet light, PTL 1 discloses, to address such a problem, a method for sterilizing even a shaded part of a sample by stirring active oxygen, generated through irradiating ozone, with ultraviolet light generated by a UV generating lamp with the use of a sterilization device having an ozone supply unit, a UV generating lamp, and a stirrer.

When the present inventors studied sterilization performance of the sterilization method according to PTL 1, there were cases where the sterilization performance was comparable to that by the conventional sterilization method using only ozone. As it is said that the sterilization performance of active oxygen exceeds intrinsically far greater than that of ozone, the inventor did not expect such a result of study.

One aspect of the present disclosure provides: a treatment device using active oxygen which can more efficiently treat the surface of a treatment target with active oxygen; and a treatment method using active oxygen which can more efficiently treat the surface of a treatment target with active oxygen.

### CITATION LIST

### Patent Literature

PTL 1 Japanese Patent Application Publication No. H1-25865

### SUMMARY OF THE INVENTION

According to at least one aspect of the present disclosure, there is provided a treatment device using active oxygen,
the treatment device comprising:
an ultraviolet transmission member; and
a first chamber and a second chamber adjacent to each other separated by the ultraviolet transmission member, wherein
the first chamber is capable of accommodating a treatment target and comprises an ozone generator,
the second chamber comprises an ultraviolet light source,
the ultraviolet light source is capable of irradiating a surface of the treatment target, which is accommodated in the first chamber, with ultraviolet light, and
a distance between the ultraviolet transmission member and the surface of the treatment target can be adjusted to 10 mm or less.

According to at least one aspect of the present disclosure, there is provided a treatment method for treating a surface of a treatment target with active oxygen, the method comprising:
(i) a step of preparing a treatment device using active oxygen, which comprises an ultraviolet transmission member, and a first chamber and a second chamber adjacent to each other separated by the ultraviolet transmission member and in which the first chamber is capable of accommodating a treatment target and comprises an ozone generator, the second chamber comprises an ultraviolet light source, the ultraviolet light source is capable of irradiating a surface of the treatment target, which is accommodated in the first chamber, with ultraviolet light, and a distance between the ultraviolet transmission member and the surface of the treatment target can be adjusted to 10 mm or less;
(ii) a step of placing, in the first chamber, the treatment target at a position to which the ultraviolet light can be emitted;
(iii) a step of filling the first chamber with ozone generated from the ozone generator; and
(iv) a step of irradiating the ozone in the first chamber with the ultraviolet light emitted by the ultraviolet light source through the ultraviolet transmission member in a state where the distance between the ultraviolet transmission member and the surface of the treatment target is 10 mm or less to generate active oxygen and treat the surface of the treatment target with the active oxygen.

According to one aspect of the present disclosure, it is possible to provide: a treatment device using active oxygen which can more efficiently treat the surface of a treatment target with active oxygen; and a treatment method using active oxygen which can more efficiently treat the surface of a treatment target with active oxygen.

Further features of the present invention will become apparent from the following description of exemplary embodiments with reference to the attached drawings.

### BRIEF DESCRIPTION OF THE DRAWINGS

FIG. 1 is a schematic cross-sectional view illustrating a configuration of a treatment device using active oxygen according to one aspect of the present disclosure.

FIG. 2 is an explanatory view of the treatment device using active oxygen of Example 1.

### DESCRIPTION OF THE EMBODIMENTS

Hereinafter, an aspect for implementing this disclosure will be specifically exemplified with reference to the drawings. However, the dimensions, materials, shapes, relative arrangements, and the like of components described in this aspect may need to be changed as appropriate depending on various conditions and the configuration of members to which the disclosure is applied. That is, it is not intended to limit the scope of this disclosure to the following aspect.

In the present disclosure, the expression of “from XX to YY” or “XX to YY” indicating a numerical range means a numerical range including a lower limit and an upper limit which are end points, unless otherwise specified. Also, when a numerical range is described in a stepwise manner, the upper and lower limits of each numerical range can be arbitrarily combined.

“Germs” as a subject of “sterilization” according to the present disclosure refers to microorganisms including animal or plant cells (including stem cells, dedifferentiated cells, and differentiated cells), tissue culture products, fused cells (including hybridomas) obtained through genetic engineering, dedifferentiated cells, and transformants (microorganisms) as well as fungi, bacteria, unicellular algae, viruses, protozoa and the like. Examples of viruses include norovirus, rotavirus, influenza virus, adenovirus, coronavirus, measles virus, rubella virus, hepatitis virus, herpes virus, and HIV virus. Examples of bacteria include Staphylococcus, Escherichia coli, Salmonella, Pseudomonas aeruginosa, Vibrio cholerae, Shigella, Bacillus anthracis, Mycobacterium tuberculosis, Clostridium botulinum, Clostridium tetani, and Streptococcus. Furthermore, examples of fungi include trichophyton, aspergillus, and candida.

In addition, active oxygen in the present disclosure includes, for example, free radicals such as hydroxyl radicals (·OH) and superoxide (·O₂⁻) produced through decomposition of ozone (O₃).

Furthermore, in the following description, components having the same functions may be denoted by the same numbers in the drawings, and description thereof may not be repeated.

According to the studies of the present inventors, the reason why the sterilization performance of the sterilization device according to PTL 1 is limited is assumed as follows.

In PTL 1, ozone is excited by irradiating it with ultraviolet light, and active oxygen with extremely high sterilization power is generated. Here, active oxygen is a general term for highly reactive oxygen active species such as superoxide anion radicals (·O₂⁻) and hydroxyl radicals (·OH), and due to the high reactivity thereof, bacteria or viruses can be instantly oxidized and decomposed.

However, since ozone absorbs ultraviolet light very well, in the sterilization device according to PTL 1, generation of active oxygen is thought to be limited to the vicinity of the UV generating lamp. That is, it is thought that ultraviolet light does not sufficiently reach ozone present at a position away from the UV generating lamp and active oxygen is less likely to be generated at a position away from the UV generating lamp.

In addition, active oxygen is very unstable with a significantly short half-life of 10⁻⁶ seconds for ·O₂⁻ and 10⁻⁹ seconds for ·OH, and is quickly converted into stable oxygen and water. For this reason, it is considered difficult to passively fill the inside of a main body of the sterilization device with active oxygen generated in the vicinity of the UV generating lamp. In other words, it is considered that sterilization through the sterilization method according to PTL 1 is substantially performed by ozone. For this reason, it is considered that the sterilization performance of the sterilization method according to PTL 1 is comparable to that by the conventional sterilization method using only ozone.

On the basis of such considerations, the present inventors have recognized that when treating a treatment target using active oxygen with a short lifespan, it is necessary to place the treatment target and the surface more actively to be treated in an active oxygen atmosphere. On the basis of this recognition, the present inventors have conducted studies and found that a treatment device using active oxygen described below allows the treatment target to be more actively placed in an active oxygen atmosphere. That is, the treatment device using active oxygen according to the present disclosure includes an ultraviolet transmission member, and a first chamber and a second chamber adjacent to each other separated by the ultraviolet transmission member and in which the first chamber is capable of accommodating a treatment target and includes an ozone generator, the second chamber includes an ultraviolet light source, and the ultraviolet light source is capable of irradiating a surface of the treatment target accommodated in the first chamber with ultraviolet light.

In the present disclosure, “treatment” of a treatment target using active oxygen includes all treatments, such as surface modification (hydrophilic treatment) of the surface of the treatment target to be treated with active oxygen, sterilization, deodorization, and bleaching, that can be achieved with active oxygen.

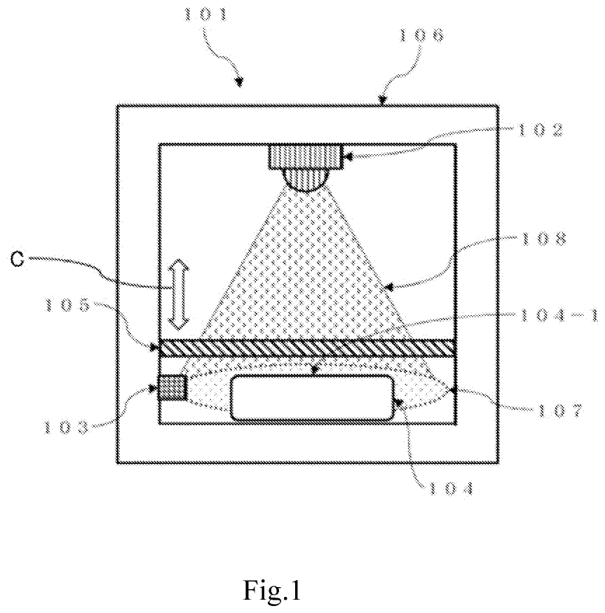

Hereinafter, a treatment device 101 using active oxygen according to one aspect of the present disclosure will be described using FIG. 1. The treatment device 101 using active oxygen according to one aspect of the present disclosure has at least an ultraviolet transmission member 105, and a first chamber and a second chamber adjacent to each other separated by the ultraviolet transmission member 105. The first chamber is capable of accommodating a treatment target 104 and includes an ozone generator 103. The second chamber includes an ultraviolet light source 102. The ultraviolet light source 102 is capable of irradiating the treatment target 104 accommodated in the first chamber with ultraviolet light 108.

That is, in the treatment device using active oxygen according to one aspect of the present disclosure, the inside of the first chamber separated by the ultraviolet transmission member 105 is filled with ozone 107 generated from the ozone generator 103 to supply the ozone 107 near a treatment surface 104-1 of the treatment target 104, and the ultraviolet light source 102 emits the ultraviolet light 108 through the ultraviolet transmission member 105 to convert the ozone 107 into active oxygen.

Here, in the treatment device using active oxygen according to one aspect of the present disclosure, the first chamber and the second chamber are separated by the ultraviolet transmission member 105, whereby suppressing diffusion of the ozone 107 generated from the ozone generator 103 comprised in the first chamber to the second chamber. For this reason, the ozone concentration in the first chamber can be increased more easily than a case where the first chamber and the second chamber are not separated by the ultraviolet transmission member. In addition, the ozone concentration in the second chamber is thought to be the same as the normal ozone concentration in dry air. By adopting such an aspect, the distance between the ultraviolet transmission member 105 and the treatment target 104 can be arbitrarily set. In addition, the ultraviolet light 108 emitted from the ultraviolet light source 102 is transmitted through the ultraviolet transmission member 105 in a state where absorption by ozone is suppressed as much as possible, and the ozone 107 is emitted on and near the surface of the treatment target 104 accommodated in the first chamber in a state where the ozone concentration is actively increased. Accordingly, active oxygen can be locally generated in a region near the treatment surface 104-1, specifically, for example, a spatial region from the treatment surface up to a height of about 1 mm (hereinafter also referred to as “surface region”) to place the treatment target more actively in an active oxygen atmosphere. For this reason, the generated active oxygen can be supplied to the surface of the treatment target before it is converted into oxygen and water. Alternatively, active oxygen can be generated in situ on a to-be-treated surface 104-1. As a result, the treatment surface 104-1 of the treatment target 104 is more reliably treated with active oxygen.

In addition, by adopting such an aspect, it is possible to suppress absorption of ultraviolet light as much as possible by ozone until the ultraviolet light 108 emitted from the ultraviolet light source 102 reaches the first chamber.

For this reason, even if the output of the ultraviolet light source is lower than that of a conventional treatment device, a sufficient amount of active oxygen can be generated for treatment of the to-be-treated surface 104-1.

Furthermore, in the treatment device according to the present disclosure, the ultraviolet transmission member 105 is preferably configured such that the distance between the ultraviolet transmission member 105 and the to-be-treated surface 104-1 of the treatment target accommodated in the first chamber is variable as shown by an arrow C. Accordingly, the distance between the to-be-treated surface 104-1 and the ultraviolet transmission member 105 can be adjusted depending on the thickness of the treatment target. The distance is preferably as short as possible within a range that allows an ozone layer to be formed on the to-be-treated surface 104-1. In the present disclosure, the distance between the surface of the treatment target 104 and the ultraviolet transmission member 105 is 10 mm or less. In a case where the thickness of the ozone layer on the to-be-treated surface 104-1 is thicker than 10 mm, ultraviolet light is consumed for decomposing ozone of the ozone layer on the ultraviolet light source side, and ozone present on a side facing the to-be-treated surface 104-1 of the ozone layer is not sufficiently decomposed. As a result, the amount of active oxygen generated on the surface of the to-be-treated surface 104-1 may be relatively reduced, and the treatment efficiency may be reduced. On the other hand, in a case where the distance is 10 mm or shorter, the in-situ generation of active oxygen on the to-be-treated surface 104-1 can be performed more reliably. As a result, the to-be-treated surface 104-1 of the treatment target 104 can be more reliably treated with active oxygen.

### <Ozone Generator>

An ozone generator is installed in a first chamber out of the first chamber and a second chamber adjacent to each other separated by an ultraviolet transmission member. Since the first chamber can accommodate a treatment target, the treatment target and the ozone generator are accommodated or installed in the same space (first chamber).

The ozone generator is not particularly limited as long as it generates ozone. Methods for generating ozone include methods using ultraviolet light, discharge, or water electrolysis, but a method using discharge is preferable to generate high- concentration ozone. In addition, examples of discharge include silent discharge, creeping discharge, and corona discharge. Specific examples of ozone generators include an ozonizer (product number: MHM500-00A) manufactured by Murata Manufacturing Co., Ltd.

In addition, the amount of ozone generated per unit time in the ozone generator without emitting ultraviolet light is preferably, for example, 5 μg/min or more. 15 μg/min or more is more preferable, 30 μg/min or more is still more preferable. The upper limit of the amount of ozone generated is not particularly limited, but is, for example, 1,000 μg/min or less or 100 μg/min or less. The amount of ozone generated can be controlled by a voltage and the like applied to the ozone generator.

The voltage applied to the ozone generator is not particularly limited, but can be, for example, 5 to 20 V DC.

### <Ultraviolet Light Source and Ultraviolet Light>

A ultraviolet light source is installed in a second chamber out of the first chamber and the second chamber adjacent to each other separated by an ultraviolet transmission member. The ultraviolet light source is not particularly limited as long as it can emit ultraviolet light that can excite ozone and generate active oxygen. In addition, the ultraviolet light source is not particularly limited as long as it has a wavelength and illuminance of ultraviolet light necessary to excite ozone and obtain an effective active oxygen concentration or effective active oxygen amount according to the purpose of a treatment.

For example, since the peak value of optical absorption spectrum of ozone is 260 nm, the peak wavelength of the ultraviolet light is preferably 220 to 310 nm, more preferably 253 to 285 nm, and still more preferably 253 to 266 nm.

As a specific ultraviolet light source, a low-pressure mercury lamp in which mercury is sealed together with an inert gas such as argon or neon in quartz glass, a cold cathode tube ultraviolet lamp (UV-CCL), an ultraviolet LED, and the like can be used. The wavelength of a low-pressure mercury lamp or a cold cathode tube ultraviolet lamp may be selected from 254 nm and the like. On the other hand, the wavelength of an ultraviolet LED may be selected from 265 nm, 275 nm and 280 nm from the viewpoint of output performance.

The illuminance of ultraviolet light on the surface of a treatment target is preferably 2.5 μW/cm² or more, more preferably 3.0 μW/cm² or more, or still more preferably 4.5 μW/cm² or more. The upper limit of the illuminance is not particularly limited, but may be, for example, 10,000 μW/cm² or less or 100 μW/cm² or less. The illuminance can be controlled by, for example, the distance between the ultraviolet light source and the surface of a treatment target or the voltage applied to the ultraviolet light source.

The voltage applied to the ultraviolet light source is not particularly limited, but can be, for example, 3 to 15 V DC.

### <Ultraviolet Transmission Member>

The ultraviolet transmission member is not particularly limited as long as it is a member that can transmit ultraviolet light, but foil (film), a sheet, a membrane, a plate, and the like that can transmit ultraviolet light can be used. As the ultraviolet transmission member, for example, biaxially oriented polypropylene (OPP), a film made of a fluororesin, a plate made of natural quartz glass or the like can be used.

The transmittance of ultraviolet light at a wavelength of 260 nm in the ultraviolet transmission member is preferably 10% or more and more preferably 50% or more. The upper limit of the transmittance is not particularly limited, but can be set to, for example, 100% or less. The thickness of the ultraviolet transmission member can be appropriately selected depending on the balance between strength and transmittance. The thickness can be set to, for example, 100 to 1,000 μm.

In addition, it is preferable that the ultraviolet transmission member can maintain sufficiently high airtightness of the second chamber. That is, it is preferable that ozone in the first chamber be prevented from passing through the ultraviolet transmission member and diffusing into the second chamber. In addition, it is preferable that the ultraviolet transmission member is attached to the inner wall of a housing to maintain sufficiently high airtightness of the second chamber. As for the airtightness of the second chamber, a C value (gap equivalent area) representing airtightness performance is preferably set to, for example, 100 cm²/m² or less. This suppresses or prevents ozone generated from the ozone generator installed in the first chamber from diffusing or entering the second chamber, and therefore ultraviolet light emitted from the ultraviolet light source installed in the second chamber can be further suppressed from being absorbed by the ozone diffused into the second chamber. For this reason, the treatment surface of a treatment target placed in the first chamber can be irradiated with ultraviolet light with higher efficiency.

Specific examples of ultraviolet transmission members include a fluororesin film (trade name: Afflex 100 N NT transparent) manufactured by AGC Inc.

### <Arrangement of Ozone Generator, Ultraviolet Light Source, and Treatment Target>

In the treatment device 101 using active oxygen, the ozone generator 103 is located in the same space (first chamber) as that of the treatment target 104, and the position thereof is not particularly limited as long as the ozone generator is placed at a position at which irradiation of the treatment target 104 with ultraviolet light 108 is not inhibited.

For example, it is preferable to bring the ozone generator 103 and the treatment target 104 close to each other so that the ozone 107 generated from the ozone generator 103 is supplied to the surface of the treatment target 104 at the shortest distance. In addition, it is also preferable to increase the ozone concentration by reducing the volume of the space (first chamber) in which the ozone generator 103 is installed as narrow as possible within a range that can accommodate the treatment target 104. The number of ozone generators 103 installed is also not particularly limited, and a plurality of ozone generators 103 may be installed to surround the treatment target 104.

The distance between the surface 104-1 of the treatment target 104 and the ultraviolet transmission member 105 which is present between the ultraviolet light source 102 and the treatment target 104 is preferably 10 mm or less. By setting the distance between the surface of the treatment target and the ultraviolet transmission member to 10 mm or less, active oxygen generated as a result of decomposition of ozone by ultraviolet light can more actively reach the treatment surface of the treatment target. On the other hand, the lower limit of the distance between the ultraviolet transmission member and the treatment target is not particularly limited, but for example, by setting it to 0.1 mm or more, it is possible to form an ozone layer on the to-be-treated surface 104-1 more easily. For this reason, the distance between the surface of the treatment target and the ultraviolet transmission member is preferably from 0.1 to 10 mm and more preferably from 1 to 3 mm.

It is preferable that the treatment device using active oxygen according to the present disclosure be adjustable so that the distance between the surface of the treatment target and the ultraviolet transmission member is 10 mm or less, for example, by placing a mounting table that can be raised and lowered in the first chamber and placing the treatment target on the mounting table.

The distance between the ultraviolet light source 102 and the ultraviolet transmission member 105 cannot be generally specified because it varies as the distance between the ultraviolet transmission member 105 and the to-be-treated surface 104-1 is adjusted, but for example, it is preferably 200 mm or less and more preferably 180 mm or less. In addition, the distance between the ultraviolet light source 102 and the ultraviolet transmission member 105 can be set to, for example, 50 mm or more.

However, it is unnecessary to place the ultraviolet transmission member at a position within 200 mm from the ultraviolet light source, and the distance between the ultraviolet light source and the ultraviolet transmission member is not particularly limited as long as active oxygen can be adjusted to an effective concentration according to the purpose of a treatment in relation to the illuminance of the ultraviolet light described above.

The distance between the surface 104-1 of the treatment target 104 and the ultraviolet light source 102 is not particularly limited, and may be any distance that can generate active oxygen to achieve an effective active oxygen concentration according to the purpose of a treatment. The closer the distance between the ultraviolet light source and the surface of the treatment target, the higher the illuminance of ultraviolet light and the more active oxygen will be generated. However, since ultraviolet light is directional, if the distance is too short, the entire surface of the treatment target may not be irradiated with the ultraviolet light. In this case, it is preferable to devise that the surface of the treatment target is uniformly irradiated with ultraviolet light by, for example, increasing the number of ultraviolet light sources or installing a reflector for reflecting ultraviolet light in at least one selected from the group consisting of the first chamber and the second chamber. In addition, it is also a preferred aspect to provide the ultraviolet light source 102 with moving means (not shown in the drawing) so that the ultraviolet light source 102 is movable so that the illuminance of the ultraviolet light is uniform.

By placing the ultraviolet light source 102 as described above, active oxygen can be locally generated in a region near the surface of the treatment target to treat the treatment target with active oxygen more efficiently.

### <Housing>

The treatment device using active oxygen of the present disclosure can comprise a housing having at least a first chamber and a second chamber with an ultraviolet transmission member as a partition wall. The first chamber is capable of accommodating a treatment target and can include an ozone generator. In addition, the second chamber may comprise an ultraviolet light source, and the ultraviolet light source can irradiate the treatment target accommodated in the first chamber with ultraviolet light.

The first chamber is preferably surrounded by a wall provided with an ultraviolet transmission member and a wall separating the outside of the treatment device from the inside of the first chamber. In addition, the wall separating the outside of the treatment device from the inside of the first chamber may be provided with means for controlling circulation of a gas between the outside of the treatment device and the inside of the first chamber. The means for controlling the circulation of a gas is not particularly limited, and examples thereof include a valve that could be opened and closed and a lid that could be opened and closed.

The size and shape of the ultraviolet transmission member and the housing, and the relative position of them with respect to the treatment target can be appropriately selected so that, for example, generated active oxygen can be actively supplied to the surface region of the treatment target in a state where the effective active oxygen concentration or effective active oxygen amount according to the purpose of a treatment is maintained.

The treatment device using active oxygen of the present disclosure can be used not only for sterilizing a treatment target, but also for general treatments implemented by supplying active oxygen to a treatment target. For example, the treatment device using active oxygen of the present disclosure can also be used for deodorizing a treatment target, bleaching a treatment target, and making a treatment target hydrophilic.

The expression “effective active oxygen concentration or effective active oxygen amount” in the present disclosure refers to an active oxygen concentration or active oxygen amount for achieving the purpose with respect to a treatment target, for example, sterilization, deodorization, bleaching, hydrophilization, or the like, and can be appropriately adjusted according to the purpose using the amount of ozone generated per unit time in an ozone generator, the illuminance and irradiation time of ultraviolet light, and the like.

### EXAMPLES

The present disclosure will be described in more detail hereinbelow with reference to Examples and Comparative Examples, but the present disclosure is not limited thereto.

### Example 1

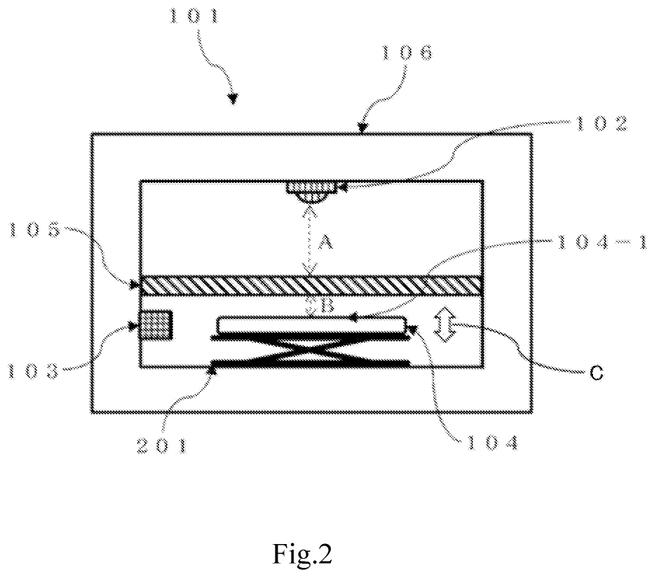

A treatment device using active oxygen of Example 1 is shown in FIG. 2. As a housing 106 of a treatment device 101 using active oxygen, a case made of ABS resin and having an inner dimension of 100 mm in height, 200 mm in width, and 200 mm in depth and a rectangular parallelepiped cross-sectional shape as shown in FIG. 2 was prepared. Next, the case was divided into two spaces by stretching an ultraviolet transmission member 105 (fluororesin film, trade name: Afflex 100N NT transparent, manufactured by AGC Inc.) parallel to the bottom surface of the housing 106 without any gap on the inner wall of the housing with an inner dimension of 25 mm in height from the bottom surface of the housing 106. In FIG. 2, the space below the ultraviolet transmission member 105 was defined as a first chamber, and the space above the ultraviolet transmission member 105 was defined as a second chamber.

In the second chamber, an ultraviolet light source 102 (UV-C LED, trade name: ZEUBE265-2CA, manufactured by Stanley Electric Co., Ltd., peak wavelength=265 nm) was placed. The distance A between the ultraviolet light source 102 and the ultraviolet transmission member 105 was 73 mm. On the other hand, a mounting table 201 on which a treatment target 104 is placed was placed in the first chamber. Since the mounting table has a lifting device for adjusting the distance B between the treatment target and the ultraviolet transmission member 105 placed thereon, the distance between the surface of the treatment target 104 and the ultraviolet transmission member 105 is configured to be variable. In addition, an ozone generator 103 (ozonizer, product number: MHM500-00A manufactured by Murata Manufacturing Co., Ltd.) was also placed in the first chamber. In this manner, a treatment device using active oxygen according to the present example was manufactured.

A light-receiving probe of an illuminometer (trade name: Spectroradiometer USR-45D, manufactured by Ushio Inc., not shown in the drawing) was placed on the mounting table 201, and the illuminance of ultraviolet light 108 was measured in a state where the distance B between the light-receiving surface of the light-receiving probe and the surface opposite to the light-receiving surface of the ultraviolet transmission member 105 was adjusted to 1 mm by adjusting the lifting device. The integrated value of a spectrum with a voltage of 7 V applied to the ultraviolet light source 102 was 4.9 μW/cm². At this time, the ozone generator 103 was not powered on so as not to be affected by shielding of the ultraviolet light 108 by ozone 107 generated from the ozone generator 103. The illuminance of the ultraviolet light 108 measured under the condition was regarded as the illuminance of the ultraviolet light 108 on the surface of the treatment target 104, which contributes to excitation of the ozone 107 on the treatment surface 104-1 of the treatment target 104.

Subsequently, to calculate the amount of ozone generated from the ozone generator 103, a hole portion (not shown in the drawing) that can be sealed with a rubber stopper was provided in the first chamber so that an internal gas can be sucked through the hole portion with a syringe. Then, one minute after applying a DC voltage of 12 V to the ozone generator 103, 100 mL of the gas inside the first chamber was collected. The collected gas was sucked into an ozone detection tube (trade name: 182SB, manufactured by Komyo Rikagaku Kogyo K.K.), and the measured ozone concentration (PPM) generated from the ozone generator 103 was measured.

The value of the measured ozone concentration was used to obtain the amount of ozone generated per unit time was obtained by the following equation.

$$Amount of ozone generated per unit time ( mg / min ) = measured ozone concentration ( PPM ) * \frac{\begin{matrix}ozone \\ molecular weight ( 48 )\end{matrix}}{22.4} * \frac{\frac{273}{273 + room temperature ( ° C . )}}{10000} * \frac{gas inside first chamber ( L )}{collected gas ( L )} = measured ozone concentration ( PPM ) * 48 / 22.4 * 273 / ( 273 + 25 ) / 10000 * 0.1 / 0.5$$

As a result, the amount of ozone generated per unit time was 67 μg/min. At this time, the ultraviolet light source 102 was not powered on so as not to be affected by decomposition of the ozone 107 by the ultraviolet light 108 emitted from the ultraviolet light source 102.

Finally, the amount of ozone generated when both the ozone generator 103 and the ultraviolet light source 102 were in operation was measured. The operating conditions for the ozone generator 103 are such that 67 μg/min of ozone is generated when only the ozone generator 103 is operated. In addition, the operating conditions for the ultraviolet light source 102 are such that the illuminance is 4.9 μW/cm² when only the ultraviolet light source 102 is operated. As a result, the amount of ozone generated when both the ozone generator 103 and the ultraviolet light source 102 were in operation was 42 μg/min. It is thought that a decrease of 25 μg/min from 67 μg/min is the amount of ozone converted into active oxygen.

### 2-1. Treatment (Hydrophilization) Test

A polypropylene resin plate (manufactured by TP Giken Co., Ltd.) with a thickness of 2 mm was cut into 30 mm in length and 30 mm in width and was placed in the center of the mounting table 201 of the treatment device 101 using active oxygen prepared in 1 above as the treatment target 104, and the height of the mounting table was adjusted so that the distance B in FIG. 2 was 1 mm. Subsequently, not only a DC voltage of 12 V was applied to the ozone generator 103 but also a DC voltage of 7 V was applied to the ultraviolet light source 102 to emit ultraviolet light, and the treatment was carried out for 1 hour. Thereafter, the contact angle of the surface of the polypropylene resin plate treated with active oxygen with respect to water was measured and compared with the contact angle before the treatment. The contact angle was measured at 23° C. and 50% RH using an automatic contact angle meter (product name: DMo-602, manufactured by Kyowa Interface Science Co., Ltd.) as a measuring device, and 0.5 μL of water was used as liquid droplets. The angle after 500 milliseconds of dropwise addition was measured, and the average value of five points was adopted. The contact angle of the surface of the polypropylene resin plate before the treatment was 102° and the contact angle thereof after the treatment was 70°. Therefore, the reduced contact angle was 32°.

### 2-2. Treatment (Sterilization) Test

An Escherichia coli sterilization test was carried out according to the following procedure. All instruments used in this sterilization test were high-pressure steam-sterilized using an autoclave. In addition, this sterilization test was conducted in a clean bench.

First, Escherichia coli (product name: “KWIK-STIK (Escherichia coli ATCC8739)” manufactured by Microbiologics) was added to an Erlenmeyer flask containing LB medium (which is 200 mL of a medium obtained by adding distilled water to 2 g of tryptone, 1 g of yeast extract, and 1 g of sodium chloride) and subjected to shaking culture at a temperature of 37° C. at 80 rpm for 48 hours. The bacterial liquid of Escherichia coli after culture was 9.2×10⁹ (CFU/mL).

0.010 mL of this bacteria liquid after culture was added dropwise onto a slide glass with a length of 3 cm, a width of 1 cm, and a thickness of 1 mm (Matsunami Glass Ind., Ltd., model number: S2441) using a micropipette and applied to the entire surface of one surface of the slide glass with the tip of the micropipette to produce Sample No. 1. Similarly, Sample No. 2 was produced.

A concave portion with a length of 3.5 cm, a width of 1.5 cm, and a depth of 2 mm was provided in the center of a plastic flat plate with a length of 30 cm, a width of 30 cm, and a thickness of 5 mm, and the above-described slide glass was installed so that the surface of the glass slide of Sample No. 1 opposite to the bacteria liquid-applied surface was in contact with the bottom surface of the concave portion. Then, the plastic flat plate on which the above-described slide glass was installed was placed on the mounting table 201 in the first chamber. Subsequently, the lifting device of the mounting table 201 was adjusted to set the distance B between the bacteria liquid-applied surface (treatment surface) of the slide glass and the surface of the ultraviolet transmission member opposite to the treatment surface to 1 mm.

Subsequently, the active oxygen treatment device was activated to treat the bacteria liquid-applied surface of the slide glass. The treatment time was 60 seconds. Next, Sample No. 1 was immersed in a test tube containing 10 mL of a buffer solution (trade name “Gibco PBS,” Thermo Fisher Scientific Inc.) for 1 hour. In order to prevent the bacteria liquid on the slide glass from drying out, the time from dropwise addition of the bacteria liquid onto the slide glass to immersion in the buffer solution was set to 60 seconds.

Next, 1 mL of a buffer solution after immersion of Sample No. 1 (hereinafter also referred to as “1/1 solution”) was placed in a test tube in which 9 mL of the buffer solution was placed to prepare a diluted solution (hereinafter referred to as “1/10 diluted solution”). A 1/100 diluted solution, a 1/1000 diluted solution, and a 1/10000 diluted solution were prepared in the same manner except that the dilution magnification with the buffer solution was changed.

Subsequently, 0.050 mL of the 1/1 solution was collected and smeared on a stamp medium (Petan Check 25 PT1025 manufactured by Eiken Chemical Co., Ltd.). This operation was repeated to produce two stamp media (n1, n2) smeared with the 1/1 solution. The two stamp media were placed in a constant temperature bath (trade name: IS600; manufactured by Yamato Scientific Co., Ltd.) and cultured at a temperature of 37° C. for 24 hours.

Similarly to the 1/1 solution, two smeared stamp media were produced and cultured for each of the diluted solutions, 1/10 diluted solution, 1/100 diluted solution, 1/1000 diluted solution, and 1/10000 diluted solution. Table 1 shows the number of colonies generated in each stamp medium for the 1/1 solution and each diluted solution. Then, the number of viable bacteria in 0.050 mL of the 1/1 solution was calculated on the basis of the number of colonies in the two stamp media relating to the 1/10 diluted solution in which the number of colonies was from 10 to 100.

Specifically, from the number of colonies observed in the stamp medium (n1) relating to the 1/10 diluted solution, the number of viable bacteria in the 1/1 solution is 24×10¹=240 (CFU). In addition, from the number of colonies observed in the stamp medium (n2) relating to the 1/10 diluted solution, the number of viable bacteria in the 1/1 solution is 18×10¹=180 (CFU). Then, the average value thereof, 210 (CFU), was taken as the number of viable bacteria in the 1/1 solution.

TABLE 1

| | n1 | n2 |
|---|---|---|
| 1/1 solution | 230 | 191 |
| 1/10 diluted solution | 24 | 18 |
| 1/100 diluted solution | 1 | 2 |
| 1/1000 diluted solution | 0 | 0 |
| 1/10000 diluted solution | 0 | 0 |

Next, regarding Sample No. 2, a 1/1 solution, a 1/10 diluted solution, a 1/100 diluted solution, a 1/1000 diluted solution, and a 1/10000 diluted solution were prepared and subjected to a culture test similarly to Sample No. 1 except that no treatment with an active oxygen supply device was performed. Then, the number of viable bacteria in the 1/1 solution relating to Sample No. 2 was calculated based on the number of colonies in two stamp media in which the number of colonies was from 10 to 100. As a result, the number of viable bacteria in 0.050 mL of the 1/1 solution according to Sample No. 2 was 595,000 (CFU).

Accordingly, the sterilization rate of Escherichia coli using the active oxygen supply device according to this test was 99.965% (=(595,000−210)/595,000]×100%).

### 2-3. Treatment (Bleaching) Test

### (1) Preparation of Samples for Bleaching Test

Chili pepper sauce (trade name: Tabasco Pepper Sauce, manufactured by McIlhenny Company) was filtered through a long fibrous non-woven fabric (trade name: Bemcot M-3II, manufactured by Asahi Kasei Corporation) to remove solids.

A paper wiper (trade name: Kimwipe S-200, manufactured by Nippon Paper Crecia Co., Ltd.) was immersed in the obtained liquid for 10 minutes. Subsequently, the paper wiper was taken out and washed with water. Washing with water was repeated until a wash liquid was no longer visually colored. Thereafter, the paper wiper was dried. Subsequently, three samples with a length of 15 mm and a width of 15 mm were cut out from the paper wiper dyed red with the chili pepper sauce.

### (2) Bleaching Test

One of the three samples was placed in the center of the mounting table 201 in the active oxygen treatment device 101, and the mounting table 201 was adjusted by being raised and lowered so that the distance between the treatment surface of the sample for a bleaching test and the ultraviolet transmission member (distance B in FIG. 2) was 1 mm. In addition, the center position of the sample 104 in the width direction (horizontal direction in FIG. 2) and the depth direction (depth direction of the paper in FIG. 2) were coincident with the center of the orientation direction of the ultraviolet light 108 emitted from the ultraviolet light source 102. Subsequently, not only a DC voltage of 12 V was applied to the ozone generator 103 but also a DC voltage of 7 V was applied to the ultraviolet light source 102 to irradiate the treatment surface with ultraviolet light for 300 minutes and to treat the to-be treated surface 104-1. This was repeated three times, and the three treated samples were compared with the untreated samples, and the degree of decolorization was visually observed and evaluated according to the following criteria.
Rank A: Completely bleached.
Rank B: Slight red color of the chili pepper sauce remained.
Rank C: Some red color of the chili pepper sauce remained.
Rank D: There was no difference in color from the part to which active oxygen was not supplied.

### 2-4. Treatment (Deodorization) Test

### (1) Preparation of Sample for Deodorization Test

A paper wiper (Kimwipe S-200, manufactured by Nippon Paper Crecia Co., Ltd.) was immersed in fabric mist (trade name: Fabric Mist Linen, manufactured by Sabon Japan Inc.) for 10 minutes, then taken out, and allowed to air dry for 6 hours. Subsequently, the paper wiper was cut into a size of 10 mm in length and 10 mm in width to obtain a sample for a deodorization test.

### (2) Deodorization Test

The sample for a deodorization test was placed in the center of the mounting table 201 in the treatment device 101 using active oxygen, and the mounting table 201 was adjusted by being raised and lowered so that the distance between the treatment surface of the sample for a deodorization test and the ultraviolet transmission member (distance B in FIG. 2) was 1 mm. In addition, the center position of the sample 104 in the width direction (horizontal direction in FIG. 2) and the depth direction (depth direction of the paper in FIG. 2) were coincident with the center of the orientation direction of the ultraviolet light 108 emitted from the ultraviolet light source 102. Subsequently, not only a DC voltage of 12 V was applied to the ozone generator 103 but also a DC voltage of 7 V was applied to the ultraviolet light source 102 to irradiate the treatment surface with ultraviolet light for 60 minutes and to treat the to-be treated surface 104-1. Then, the extent to which an odor of the treated sample remained in comparison with a sample that had not been treated with active oxygen was evaluated according to the following intensity criteria. The evaluation was conducted on 5 subjects, and the strength criteria selected by at least 3 subjects were adopted.
Rank A: Odorless
Rank B: Barely detectable odor (detection threshold value)
Rank C: Weak odor that can be recognized as fabric mist odor (recognition threshold value)
Rank D: There was no difference from untreated sample.

### Example 2

A treatment device using active oxygen was manufactured and evaluated in the same manner as in Example 1 except that the voltage of the ultraviolet light source 102 of Example 1 was reduced from 7 V DC to 4 V DC to lower the illuminance of ultraviolet light.

### Example 3

A treatment device using active oxygen was manufactured and evaluated in the same manner as in Example 1 except that the ozone generator of Example 1 was changed to a scorotron corona charger (DC −5 kV applied) used as a charging member in an electrophotographic device.

### Example 4

A treatment device using active oxygen was manufactured and evaluated in the same manner as in Example 1 except that the ultraviolet light source in Example 1 was changed to UV-C LED (model number: LJU1106EAE-275-TR, manufactured by Stanley Electric Co., Ltd., peak wavelength=275 nm).

### Examples 5 and 6

Treatment devices using active oxygen were manufactured and evaluated in the same manner as in Example 1 except that the distance B between the treatment surface and the surface opposite to the treatment surface of the ultraviolet transmission member was changed as shown in Table 2.

### Comparative Examples 1 to 3

The conditions of Comparative Examples 1 to 3 were the same as those of Example 1 except for the following configurations.

Comparative Example 1: The ultraviolet transmission member was removed.

Comparative Example 2: No voltage was applied to the ozone generator, but a voltage was applied to the ultraviolet light source.

Comparative Example 3: A voltage was applied to the ozone generator, but no voltage was applied to the ultraviolet light source.

### Comparative Example 4

A treatment device using active oxygen was manufactured and evaluated in the same manner as in Example 1 except that the distance B between the treatment surface and the surface opposite to the treatment surface of the ultraviolet transmission member was changed as shown in Table 2.

TABLE 2

| | | | | | | | Hydrophilization | Sterilization test | | | |
|---|---|---|---|---|---|---|---|---|---|---|---|
| | | Ultraviolet | UV | Dis- | Amount of | | test | Number | | | |
| | | trans- | peak | tance | ozone | UV | Reduced | of viable | Steril- | Deodorization | Bleaching |
| | | mission | wavelength | B | generated | Illuminance | contact | bacteria | ization | test | test |
| | | member | (nm) | (mm) | (μg/min) | (μW/cm²) | angle (°) | (CFU) | rate | Evaluation rank | |
| Examples | 1 | Present | 265 | 1 | 67 | 4.9 | 32 | 210 | 99.965% | A | A |
| | 2 | Present | 265 | 1 | 67 | 2.6 | 27 | 1050 | 99.824% | A | A |
| | 3 | Present | 265 | 1 | 5 | 4.9 | 24 | 1800 | 99.697% | A | A |
| | 4 | Present | 275 | 1 | 67 | 4.6 | 31 | 570 | 99.904% | A | A |
| | 5 | Present | 265 | 3 | 67 | 4.9 | 22 | 2150 | 99.639% | B | B |
| | 6 | Present | 265 | 10 | 67 | 4.9 | 15 | 6300 | 98.941% | B | B |
| Comparative | 1 | Absent | 265 | 1 | 67 | 4.9 | 5 | 375000 | 36.975% | C | D |
| Examples | 2 | Present | 265 | 1 | 0 | 4.9 | 0 | 84000 | 85.882% | D | D |
| | 3 | Present | 265 | 1 | 67 | 0 | 8 | 260000 | 56.303% | C | C |
| | 4 | Present | 265 | 15 | 67 | 4.9 | 7 | 102000 | 82.857% | C | C |

In Table 2, the amount of ozone generated indicates the amount of ozone generated when the ultraviolet light source was not powered on.

As shown in Table 2, the contact angle did not decrease when exposed to ultraviolet light as shown in Comparative Example 2. In addition, the contact angle decreased when ozone was generated as shown in Comparative Example 3. Furthermore, when both ozone generation and ultraviolet irradiation were performed, the contact angle further decreased due to high reactivity of active oxygen.

In Comparative Example 3, some effects of sterilization, deodorization, and bleaching by ozone were observed, but they were not as good as Examples 1 to 6. In Comparative Example 2, some effect of sterilization by ultraviolet light was observed, but no effects of deodorization and bleaching were observed. In Comparative Example 1, since there was no ultraviolet transmission member, ultraviolet light decomposed ozone, and ozone absorbed ultraviolet light. Therefore, the sterilization effect was even inferior to that in Comparative Examples 2 and 3 which were treated with ultraviolet light or ozone alone.

In Comparative Example 4, since the distance B between the treatment target and the ultraviolet transmission member was 15 mm, the amount of active oxygen generated on the treatment surface of the treatment target was smaller than that in Example 6, and the treatment efficiency of the treatment surface decreased significantly. As a result, compared to Example 6, the sterilization, deodorization, and bleaching effects were inferior.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

What is claimed is:

1. A treatment device using active oxygen,
the treatment device comprising:
an ultraviolet transmission member; and
a first chamber and a second chamber adjacent to each other separated by the ultraviolet transmission member, wherein
the first chamber is capable of accommodating a treatment target and comprises an ozone generator,
the second chamber comprises an ultraviolet light source,
the ultraviolet light source is capable of irradiating a surface of the treatment target, which is accommodated in the first chamber, with ultraviolet light, and
a distance between the ultraviolet transmission member and the surface of the treatment target can be adjusted to 10 mm or less.

2. The treatment device using active oxygen according to claim 1, wherein
the distance between the ultraviolet transmission member and the surface of the treatment target accommodated in the first chamber is configured to be variable.

3. The treatment device using active oxygen according to claim 1, wherein
the first chamber is surrounded by a wall provided with the ultraviolet transmission member and a wall separating the outside of the treatment device from the inside of the first chamber.

4. The treatment device using active oxygen according to claim 3, wherein
the wall separating the outside of the treatment device from the inside of the first chamber is provided with means for controlling circulation of a gas between the outside of the treatment device and the inside of the first chamber.

5. The treatment device using active oxygen according to claim 1, wherein
the ultraviolet light emitted by the ultraviolet light source has a peak wavelength of 220 to 310 nm.

6. The treatment device using active oxygen according to claim 1, wherein
the ultraviolet light on the surface of the treatment target has an illuminance of 2.5 μW/cm² or more.

7. The treatment device using active oxygen according to claim 1, wherein
an amount of ozone generated in the ozone generator per unit time without irradiation of the ultraviolet light is 5 μg/min or more.

8. A treatment method for treating a surface of a treatment target with active oxygen, the method comprising:
(i) a step of preparing a treatment device using active oxygen, which comprises an ultraviolet transmission member, and a first chamber and a second chamber adjacent to each other separated by the ultraviolet transmission member and in which the first chamber is capable of accommodating a treatment target and comprises an ozone generator, the second chamber comprises an ultraviolet light source, the ultraviolet light source is capable of irradiating a surface of the treatment target, which is accommodated in the first chamber, with ultraviolet light, and a distance between the ultraviolet transmission member and the surface of the treatment target can be adjusted to 10 mm or less;
(ii) a step of placing, in the first chamber, the treatment target at a position to which the ultraviolet light can be emitted;
(iii) a step of filling the first chamber with ozone generated from the ozone generator; and
(iv) a step of irradiating the ozone in the first chamber with the ultraviolet light emitted by the ultraviolet light source through the ultraviolet transmission member in a state where the distance between the ultraviolet transmission member and the surface of the treatment target is 10 mm or less to generate active oxygen and treat the surface of the treatment target with the active oxygen.

9. The treatment method using active oxygen according to claim 8, wherein
the distance between the ultraviolet transmission member and the surface of the treatment target accommodated in the first chamber is configured to be variable.
